(19) United States
(12) Patent Application Publication
(10) Pub. No.: US 2024/0268948 A1
(43) Pub. Date: Aug. 15, 2024

(54) FENESTRATED ENDOPROSTHESIS OF THE THORACIC AORTA

(71) Applicants: UNIVERSITE CLAUDE BERNARD LYON 1, VILLEURBANNE (FR); CENTRE NATIONAL DE LA RECHERCHE SCIENTIFIQUE, PARIS (FR); INSTITUT NATIONAL DE LA SANTE ET DE LA RECHERCHE MEDICALE, PARIS (FR); UNIVERSITE JEAN MONNET SAINT ETIENNE, SAINT-ETIENNE (FR); HOSPICES CIVILS DE LYON, LYON (FR)

(72) Inventor: Benoit COSSET, LYON (FR)

(73) Assignees: UNIVERSITE CLAUDE BERNARD LYON 1, VILLEURBANNE (FR); CENTRE NATIONAL DE LA RECHERCHE SCIENTIFIQUE, PARIS (FR); INSTITUT NATIONAL DE LA SANTE ET DE LA RECHERCHE MEDICALE, PARIS (FR); UNIVERSITE JEAN MONNET SAINT ETIENNE, SAINT-ETIENNE (FR); HOSPICES CIVILS DE LYON, LYON (FR)

(21) Appl. No.: 18/567,531

(22) PCT Filed: Jun. 7, 2022
(86) PCT No.: PCT/EP2022/065404

§ 371 Date: Dec. 6, 2023

(30) Foreign Application Priority Data
Jun. 10, 2021 (FR) FR2106128

(51) Int. Cl. A61F 2/07; A61F 2/06; A61F 2/89

(52) U.S. Cl. CPC A61F 2/07; A61F 2/89; A61F 2002/061

(57) ABSTRACT

A fenestrated endoprosthesis of the thoracic aorta including a first mesh of stents, a main tubular endoprosthesis made of fabric, the main tubular endoprosthesis being rigidly attached to the first mesh, and having an arcuate central portion configured to be received in the aortic arch of a patient, the arcuate central portion extending between a first and a second end, each configured to be received respectively in a portion of the ascending or descending aorta, respectively. The fenestrated endoprosthesis further includes a flange also made of fabric and associated with a second mesh of stents, the flange having a substantially frustoconical shape and having a base, the base of the flange being mounted on an opening made in the upper face of the arcuate central portion, the flange being configured to be received at the base of the supra-aortic trunks of the patient.

### FIELD

The present invention is concerned with the field of endoprostheses, and more particularly with thoracic aortic endoprostheses.

### BACKGROUND

An aneurysm is a localised dilation of the wall of an artery. An aneurysm occurs when there is a loss of parallelism between the edges of the vessel and its gauge is greater than 1.5 times normal.

The main risk is rupture of the aneurysm, which is usually fatal.

While all arteries can be affected, the aorta is frequently impacted. It is the largest artery in the human body.

All aortic aneurysms inexorably increase in size over time, and the risk of rupture becomes significant once the diameter of the aorta exceeds 5 cm.

However, if an aneurysm is detected in time, it is possible to prevent its rupture by open surgery, the indication being clear when the diameter exceeds 5.5 cm. However, this type of thoracic surgery is associated with a high morbidity and mortality rate. Since the early 1990s, a less invasive treatment has been provided for aneurysms of the descending aorta, involving placement of an endoprosthesis from the femoral artery: endovascular repair.

Aortic dissection corresponds to a breach in the wall of the aorta, with blood infiltrating the wall and dividing it in two. This creates two channels. Aortic dissection is a serious condition that affects three people per 100,000 inhabitants. It can affect the ascending aorta, the descending aorta or both. When it affects the ascending aorta, the standard treatment is emergency replacement of the aorta by open surgery. In the descending aorta, the standard treatment is medical treatment alone. In the event of complications, endovascular treatment using an endoprosthesis is performed.

The endoprosthesis is a stent (that is, a meshed tubular metal device, commonly known as a “spring”) associated with a sealed wall (also known as a “stent graft” or “covered stent” in French), unfolded inside the artery at the site of the aneurysm. It isolates the latter from the blood flow and recreates a cylindrical artery section in its place.

The aorta is divided into two parts, the ascending aorta at the thoracic level and the descending aorta at the thoracic and abdominal levels.

Endovascular treatment of aneurysms and dissections of the descending aorta has become the standard treatment.

However, for the ascending aorta, open surgery remains the standard treatment. This is mainly due to the anatomical complexities of the zone: the aorta takes the form of a crozier and is surrounded by noble vessels that cannot tolerate obliteration: coronary ostia, supra-aortic trunks vascularising the brain.

International patent application WO2007/028086 describes a thoracic endoprosthesis adapted to the aortic arch by virtue of the presence of “windows” in the wall of the stent facing the three vessels, these windows allowing blood to pass through the prosthesis and thus vascularise the vessels going to the head, neck and arms.

However, the insertion of this stent is a tricky operation, since it has to be ensured that it does not rotate during insertion, otherwise the windows will not be placed facing the vessels and may be totally or partially obstructed, which would be fatal for the patient.

Additionally, the time taken to deploy the endoprosthesis is still between three and six hours, which is a heavy operation, performed under general anaesthetic. The complexity of the procedure means that it has to be performed in expert centres by experienced operators, thereby limiting the spread and democratisation of this treatment.

The operation requires placing guides in the vessels of the neck, and is therefore associated with a high risk of cerebrovascular accident (CVA). There is also the problem of anchoring the endoprosthesis to avoid its movement in the direction of the blood flow.

Finally, these types of endoprostheses need to be custom-built from the patient's CT scan and therefore require a long delivery time (an average of one month), which is incompatible with the emergency treatment of these pathologies.

Document WO 2011/041773 A1 is directed to an endoprosthesis with two lateral branches to be inserted into the supra-aortic trunks. This type of endoprosthesis also needs to be custom-built from the patient's CT scan and requires a significant amount of operation time to be placed into the patient's body, since the branches also need to be inserted into the supra-aortic trunks. The complexity of the procedure requires implantation in expert centres by experienced operators, thereby limiting spread and democratisation of this treatment.

Document US 2011/270380 A1 describes an endovascular prosthesis that comprises a tubular body and an outer connector extendable from a folded configuration against the tubular body to a deployed position.

As with the previous document, this type of endoprosthesis requires a customised design and has a significant operation time due to the ramifications it has. The complexity of the procedure requires implantation in expert centres by experienced operators, thus limiting spread and democratisation of this treatment.

### SUMMARY

To this end, the aim of the present invention is to enable aneurysms and dissections of the ascending aorta, including the aortic arch, to be treated at the thoracic level with maximum safety, while reducing complexity of the surgical procedure.

More particularly, one object of the invention is a fenestrated endoprosthesis of the thoracic aorta including a first stent meshing, a main tubular endoprosthesis made of fabric, said main tubular endoprosthesis being integral with said first stent meshing, and having an arcuate central part configured to be housed in the aortic arch of a patient, said arcuate central part extending between a first end configured to be housed in a portion of the ascending aorta and a second end configured to be housed in a portion of the descending aorta, the fenestrated endoprosthesis of the thoracic aorta further including a flange also made of fabric and associated with a second stent meshing, said flange being substantially frustoconical in shape and provided with a base, said base of the flange being fitted over an aperture made on the upper face of the arcuate central part of the main tubular endoprosthesis, said flange being configured to be housed at the base of the patient's supra-aortic trunks.

Optional, complementary or alternative characteristics of the invention are set out below.

According to some characteristics, the second stent meshing may consist mainly of a mesh extending over the entire circumference of the flange in substantially sinusoidal circumvolutions, the radial force of said meshing making it possible to press the flange against the wall of the base of the supra-aortic trunks and thus reduce the risk of endoleaks.

According to other characteristics, the second stent meshing may consist mainly of a plurality of disjointed meshes radially offset from one another, extending over the entire circumference of the flange in substantially sinusoidal circumvolutions, the radial force of said meshing making it possible to press the flange against the wall of the base of the supra-aortic trunks and thus reduce the risk of endoleaks.

According to other characteristics, the second stent meshing may further include an elliptical mesh or a mesh defining the contour of a hyperbolic paraboloid surface, at the base of the flange, so as to maintain the aperture made on the upper face of the arcuate central part of the main tubular endoprosthesis.

According to yet other characteristics, the first stent meshing may consist mainly of several disjointed meshes, each extending circularly over the entire circumference of the main tubular endoprosthesis in substantially sinusoidal circumvolutions, the radial force of said meshing making it possible to press the main tubular endoprosthesis against the aortic wall and thus reduce the risk of endoleaks.

According to yet other characteristics, the first stent meshing may include at least one mesh attached to the second stent meshing, so as to prevent plications of the main tubular endoprosthesis. The plications correspond to folds formed by the body of the endoprosthesis when the prosthesis is incorrectly deployed or when there are significant outer stresses, and can lead to poor blood flow from simple stenosis to occlusion of the prosthesis.

According to yet other characteristics, the first stent meshing may include at least one mesh attached to the mesh at the base of the flange, so as to prevent plications of the main tubular endoprosthesis.

Advantageously, at least one of the ends of the main tubular endoprosthesis or the free end of the flange can be an apparent meshing zone devoid of fabric.

The fabric covering the stent meshing can preferably be selected from the list defined by braided polyester, braided Dacron and PTFE.

The stent meshings can preferably be made of a shape memory alloy, preferably a Nickel-Titanium alloy.

Advantageously, the fenestrated endoprosthesis of the thoracic aorta may comprise radiopaque markers, preferably disposed at the ends of the main tubular endoprosthesis and/or at the free end of the flange.

According to one particular embodiment, the first meshing may project onto the flange.

According to another particular embodiment, the second meshing may project onto the main tubular endoprosthesis.

Advantageously, a reinforcement may extend along the convex face of the arcuate central part of the main tubular endoprosthesis, and even more advantageously over the entire length of said main tubular endoprosthesis.

Preferably, this reinforcement is made of nitinol or suture wire.

### BRIEF DESCRIPTION OF THE DRAWINGS

Further advantages and features of the invention will become apparent upon reading the detailed description of implementations and embodiments which are by no means limiting, and the following appended drawings:

FIG. 1 This figure represents a schematic side view of a thoracic endoprosthesis according to one particular embodiment of the invention.

FIG. 2 This figure represents a schematic side view of a thoracic endoprosthesis placed in the aortic arch of a patient according to one particular embodiment of the invention.

FIG. 3 This figure represents a schematic top view of a thoracic endoprosthesis according to one particular embodiment of the invention.

FIG. 4 This figure represents a schematic side view of a thoracic endoprosthesis according to the invention, with dimensions.

FIG. 5 This figure represents a schematic top view of a thoracic endoprosthesis according to the invention, with dimensions.

FIG. 6 This figure represents a schematic side view of a patient's aortic arch equipped with a thoracic endoprosthesis according to one particular embodiment of the invention.

FIG. 7 This figure represents a schematic side view of a thoracic endoprosthesis according to one particular embodiment of the invention.

FIG. 8 This figure represents a schematic top view of a thoracic endoprosthesis according to one particular embodiment of the invention.

FIG. 9 This figure represents a schematic side view of a thoracic endoprosthesis placed in the aortic arch of a patient according to one particular embodiment of the invention.

### DETAILED DESCRIPTION

The embodiments described below being by no means limiting, it will be possible especially to consider alternatives to the invention comprising only a selection of characteristics described, isolated from the other characteristics described even if this selection is isolated within a sentence comprising these other characteristics, if this selection of characteristics is sufficient to confer a technical advantage or to differentiate the invention from information in prior art.

This selection comprises at least one characteristic, preferably functional without structural details, or with only part of the structural details if this part alone is sufficient to confer a technical advantage or to differentiate the invention from information in prior art.

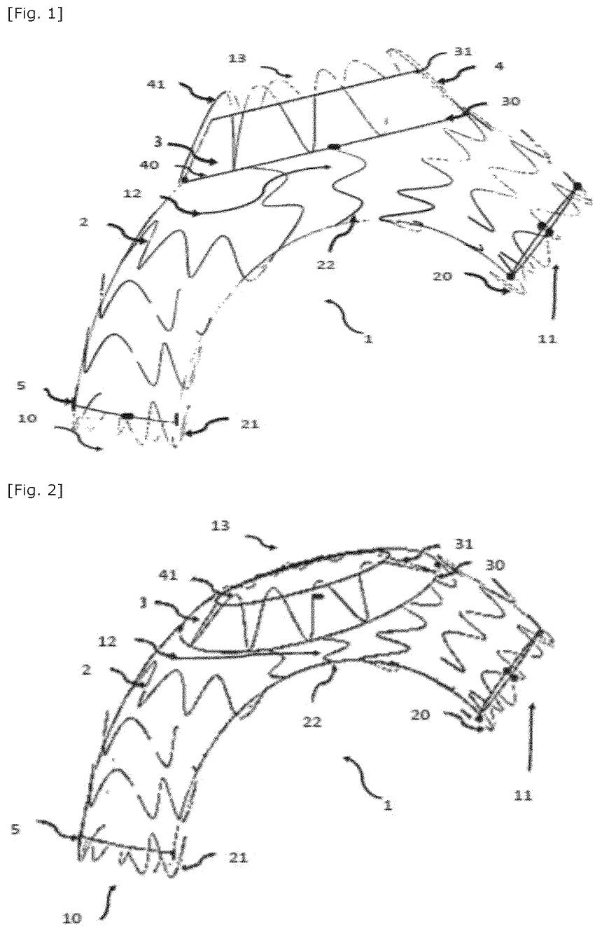

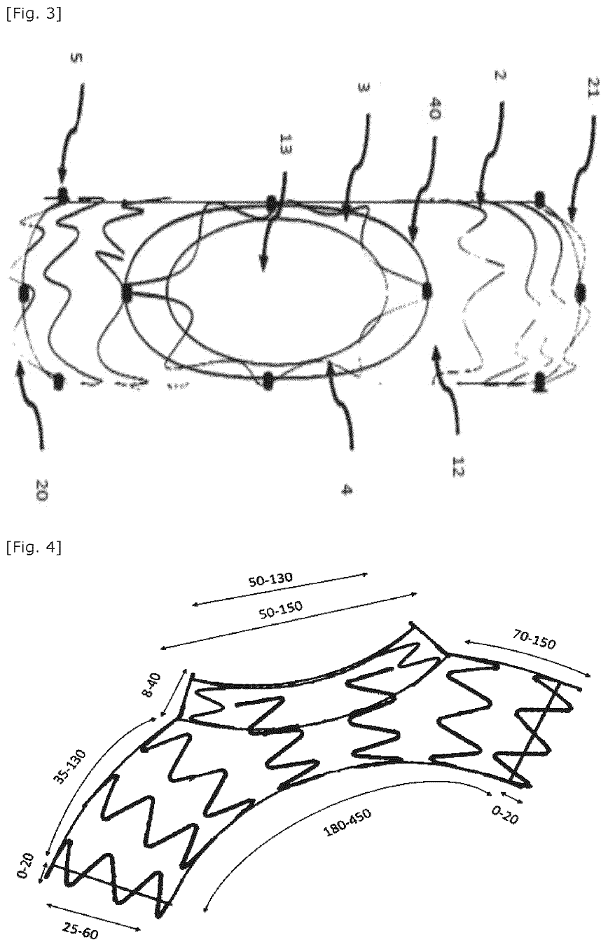

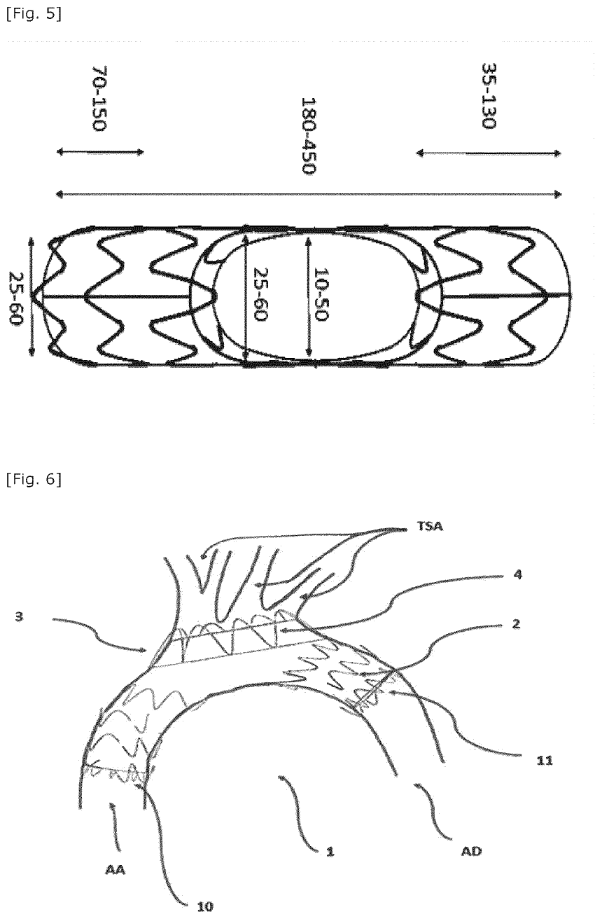

According to the invention and as represented in FIG. 1 and in FIG. 6, the fenestrated endoprosthesis of the thoracic aorta includes a first stent meshing 2 and a main tubular endoprosthesis 1 made of biocompatible fabric forming a sealed jacket, the main tubular endoprosthesis 1 recreating a cylindrical vessel and isolating the sac formed by the aneurysm from the blood circulation.

The main tubular endoprosthesis has an arcuate central part 12 configured to be housed in the aortic arch of a patient. This arcuate central part 12 extends between a first end 10 configured to be housed in a portion of the ascending aorta AA and a second end 11 configured to be housed in a portion of the descending aorta DA.

Advantageously, the fabric constituting the main tubular endoprosthesis can be selected from the list defined by braided polyesters, braided Dacron and PTFE.

This fabric may, for example, be PTFE such as laminated Teflon® or Dacron®, these materials being commonly used in simple endoprostheses.

Furthermore, the external diameter and length of the unfolded main tubular endoprosthesis are advantageously selected to be between 25 and 60 mm and between 180 and 450 mm respectively. These dimensions enable the main tubular endoprosthesis 1 to be perfectly adapted to the morphology of the aortic arch (also known as the transverse aorta) of patients.

However, the invention is not limited to particular dimensions or materials. The person skilled in the art will be able to adapt it to each case.

According to the invention, the main tubular endoprosthesis 1 includes an aperture 13 on the upper face of its arcuate central part 12.

By “upper face”, it is meant the convex portion of the arcuate central part 12, that is, that which faces the base of the supra-aortic trunks.

According to the invention, the fenestrated endoprosthesis of the thoracic aorta further includes a flange 3 also made of fabric and associated with a second stent meshing 4.

This flange 3 is configured to be housed at the base of the patient's supra-aortic trunks TSA.

By “housed at the base of the supra-aortic trunks”, it is meant that the flange faces the supra-aortic trunks and therefore does not engage in any of the supra-aortic trunks.

It is precisely this position of the flange at the main aorta that allows “universality” of the endoprosthesis as well as simplicity and quickness of implantation.

The flange 3 is substantially frustoconical in shape and its base 30 may be substantially elliptical.

The base of the flange can also advantageously define the contour of a hyperbolic paraboloid surface. This geometry allows the widest possible aperture of the flange, which makes the flange compatible with most anatomical variations in the supra-aortic trunks. In other words, the three-dimensional nature of the hyperbolic paraboloid surface allows maximum enlargement of the aperture 13 in the three planes of space: antero-posterior, lateral, supero-inferior. Advantageously, the dimensions of the major axes at the base and the apex of the frustoconical flange 3 are selected to be between 50 and 150 mm and between 50 and 130 mm respectively, in order to adapt perfectly to the morphology of the patient's aortic arch.

Similarly, the dimensions of the minor axes at the base and the apex of the frustoconical flange 3 are selected to be between 25 and 60 mm and between 10 and 50 mm respectively, in order to adapt perfectly to the morphology of the patient's aortic arch.

The frustoconical flange 3 preferably extends over a length of between 8 and 40 mm.

However, the invention is not limited to particular dimensions or materials. The person skilled in the art will be able to adapt it to specific cases.

The base of the flange 3 is fitted over the aperture 13 made on the upper face of the arcuate central part 12 of the main tubular endoprosthesis 1.

By fitted over, it is meant that the flange 3 is attached to the main tubular endoprosthesis 1 at the aperture 13.

The assembly formed by the main tubular endoprosthesis and the flange can be made as a single unitary piece, for example by 3D printing.

Alternatively, the flange 3 can be sutured to the main tubular endoprosthesis 1 by bonding or stitching, for example, the aperture 13 being drilled in the main tubular endoprosthesis 1 before the suture. The suture has to ensure sealing of the junction between the main tubular endoprosthesis 1 and the flange 3.

The flange 3 preferably consists of the same fabric as the main tubular endoprosthesis 1.

The main tubular endoprosthesis 1 is associated with the first stent meshing 2, in that the stents of the first meshing are attached generally to the outer face of the main tubular endoprosthesis, so that the stents maintain the main tubular endoprosthesis in its required shape.

Other non-limiting configurations in which the stents are impregnated into the tissue or disposed against the inner face of the main tubular endoprosthesis 1 are also contemplatable.

Preferably, the stents are made of a shape memory alloy, such as Nickel-Titanium alloys and in particular Nitinol.

According to other non-limiting configurations, the stents can be Z-Stents® made of a chromium and cobalt alloy, for example.

Advantageously, the first stent meshing 2 may consist mainly of several disjointed meshes, each extending circularly over the entire circumference of the main tubular endoprosthesis 1.

Preferably, these meshes may extend in substantially sinusoidal or zigzag-shaped circumvolutions, so that the meshes deform like springs.

Thus, the radial force of said meshing makes it possible to press the main tubular endoprosthesis 1 against the aortic wall and reduce the risk of endoleaks.

According to other non-limiting configurations, the stents can be connected in pairs by a plurality of connecting means. The connecting means may be the subject of numerous embodiments, but generally may consist of metal bars (for example also made of a Chromium and Cobalt alloy, or Nitinol) fixed at their two ends to the structure of the stents, for example by means of a loop or hook surrounding a mesh of a stent.

Regarding the flange 3, this is associated with the second stent meshing 4, in that the stents of the second meshing are attached generally to the outer face of the flange, so that the stents maintain the flange in its required shape.

Other non-limiting configurations in which the stents are impregnated into the tissue or disposed against the inner face of the main tubular endoprosthesis 1 are also contemplatable.

Preferably, the second stent meshing 4 mainly consists of a single mesh, which extends over the entire circumference and over the entire length of the flange 3.

Preferably, this mesh can extend in substantially sinusoidal or zigzag-shaped circumvolutions, so that the mesh deforms like a spring.

Thus, the radial force of the second stent meshing 4 makes it possible to press the flange 3 against the wall of the base of the supra-aortic trunks TSA and reduce the risk of endoleaks.

As represented in FIG. 2 and FIG. 6, it is seen that the wall at the base of the supra-aortic trunks TSA tends to exert pressure on the flange 3, which in turn exerts counter-pressure against the wall at the base of the supra-aortic trunks, by virtue of the second stent meshing 4.

According to one alternative embodiment, the second stent meshing 4 mainly consists of two disjointed meshes radially offset from one another, so as to reinforce support of the flange produced by the two stent meshes.

Preferably, the stents of the second meshing are made of a shape memory alloy, such as, for example, Nickel-Titanium alloys and in particular Nitinol.

According to other non-limiting configurations, the stents may be Z-Stents® made of a chromium and cobalt alloy, for example.

Advantageously, the second stent meshing 4 may further include an elliptical mesh 40 at the base 30 of the flange 3, so as to maintain the aperture 13 made on the upper face of the arcuate central part 12 of the main tubular endoprosthesis 1.

For the same purpose, the second stent meshing 4 may further include a mesh defining the contour 40′ of a hyperbolic paraboloid surface.

Also advantageously, the first stent meshing 2 may include at least one mesh 22 attached to the second stent meshing 4, so as to prevent plications of the main tubular endoprosthesis 1.

This mesh 22 may preferably be attached to the elliptical mesh 40 or to the mesh 40′ defining the contour of a hyperbolic paraboloid surface of the base 30 of the flange 3, so as to prevent plications of the main tubular endoprosthesis 1.

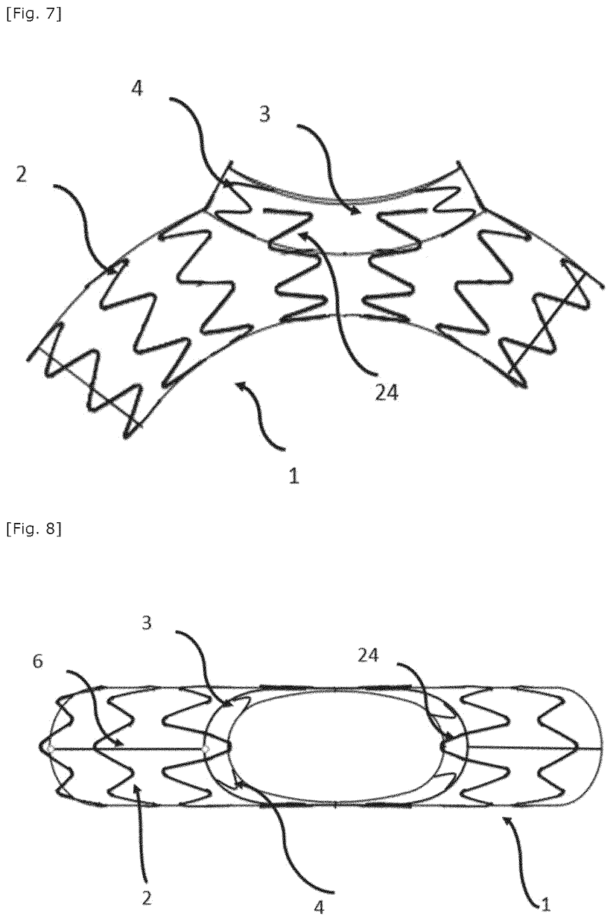

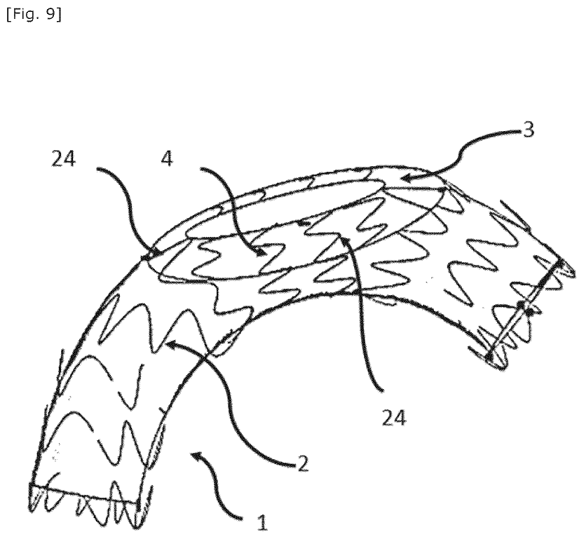

As represented in FIGS. 7, 8 and 9, and in order to maintain the flange 3 as vertical as possible and thus prevent it from collapsing, the first meshing 2 advantageously projects onto the flange 3 over portions 24 of the flange, in order to reinforce the deployment of the flange against the supra-aortic trunks.

More particularly, some meshes of the first meshing extend to the flange so as to form an incomplete circle. Their presence makes it possible to limit the secondary meshing on the flange and thus decrease the overall size of the fenestrated endoprosthesis. The endoprosthesis is thus miniaturised, making it possible to use smaller diameter launchers which can be introduced into femoral arteries of smaller gauge.

Of course, as an alternative and for the same purpose, the second meshing 4 may also project onto the main tubular endoprosthesis 1 according to this same principle.

Advantageously, a reinforcement 6 extends along the convex face of the arcuate central part 12 of the main tubular endoprosthesis 1, and preferably over the entire length of said main tubular endoprosthesis. This reinforcement thus makes it possible to avoid “accordion” type plications in the longitudinal direction, and consequently promotes deployment of the endoprosthesis over its entire length.

This reinforcement 6 may, for example, be made of nitinol or consisting of suture wire on the fabric.

So as to facilitate anchoring of the main tubular endoprosthesis, one of the ends of the main tubular endoprosthesis may be an apparent meshing zone 20, 21 devoid of tissue, which zone is called “free flow”.

Similarly, the free end 31 of the flange 3 may be an apparent meshing zone 41 devoid of tissue.

These apparent meshing zones 20, 21 and 41 in fact consist of a mesh extending in substantially sinusoidal or zigzag-shaped circumvolutions. The apices of the sinusoidal curve or of the zigzags can then be brought closer or moved apart along the radial direction, that is the direction perpendicular to that which corresponds to the flow of the blood flow.

These apparent meshing zones 20, 21 and 41 extend over a distance of less than or equal to 20 mm.

The apices can be brought closer together along the radial direction when the fenestrated endoprosthesis is placed in the thoracic aorta by means of a cone and a delivery guide.

Once positioning in the aorta is complete, the mesh tends to return to its initial shape and the apices move apart along the radial direction to anchor in the wall of the ascending aorta AA, in the wall of the descending aorta AD and in the wall of the base of the supra-aortic trunks TSA.

So as to facilitate insertion of the fenestrated endoprosthesis of the thoracic aorta, the latter may comprise radiopaque markers 5, preferably disposed at the ends of the main tubular endoprosthesis 1 and/or at the free end 31 of the flange 3.

The invention thus makes it possible to optimise anchoring of the fenestrated endoprosthesis by avoiding movement of the prosthesis in the direction of the flow.

This is especially reinforced by:
The apparent meshing zones (also called free flow) at the ends of the prosthesis and at the flange.
The first and second stent meshings oriented perpendicular to each other.
The length of the endoprosthesis, which is greater than that of a conventional endoprosthesis (10 to 20 cm for the Gore TAG endoprosthesis, for example, which is the most commonly used for aneurysms and dissections of the descending thoracic aorta).
The flange 3 and the second stent meshing 4 associated therewith tend to deploy against the base of the wall of the supra-aortic trunks.

This fenestrated endoprosthesis makes it possible to overcome a large number of anatomical features of patients which contraindicate endovascular treatment with existing endoprostheses (insufficient diameter of the TSAs, insufficient length of the TSAs, particular location of the TSAs on the aorta, TSAs too closely related to each other, etc.).

The flange 3 and the second stent meshing 4 associated therewith make it possible to avoid the creation of aortic struts which make deployment longer and more technical due to the catheterisation of the TSAs which consists in placing guides in a vessel, and more at risk of major complications, such as cerebrovascular accidents.

This fenestrated endoprosthesis allows for rapid intervention due to the ease with which the endoprosthesis can be deployed, that is less than one hour compared with the usual three to six hours.

It should be noted that the different characteristics, forms, alternatives and embodiments of the invention may be associated with one another, in various combinations, insofar as they are not incompatible or mutually exclusive.

1-14. (canceled)

15. A fenestrated endoprosthesis of the thoracic aorta comprising a first stent meshing, a main tubular endoprosthesis made of fabric, said main tubular endoprosthesis being integral with said first stent meshing, and having an arcuate central part configured to be housed in the aortic arch of a patient, said arcuate central part extending between a first end configured to be housed in a portion of the ascending aorta and a second end configured to be housed in a portion of the descending aorta, wherein the fenestrated endoprosthesis of the thoracic aorta further includes a also made of fabric and associated with a second stent meshing, said flange being substantially frustoconical in shape and provided with a base, said base of the flange being fitted over an aperture made on the upper face of the arcuate central part of the main tubular endoprosthesis, said flange being configured to be housed at the base of the patient's supra-aortic trunks.

16. The fenestrated endoprosthesis of the thoracic aorta according to claim 15, wherein the second stent meshing mainly consists of a mesh extending over the entire circumference of the flange in substantially sinusoidal or zigzag circumvolutions, the radial force of said meshing making it possible to press the flange against the wall of the base of the supra-aortic trunks and thus reduce the risk of endoleaks.

17. The fenestrated endoprosthesis of the thoracic aorta according to claim 15, wherein the second stent meshing mainly consists of a plurality of disjointed meshes radially offset from one another, extending over the entire circumference of the flange in substantially sinusoidal or zigzag circumvolutions, the radial force of said meshing making it possible to press the flange against the wall of the base of the supra-aortic trunks and thus reduce the risk of endoleaks.

18. The fenestrated endoprosthesis of the thoracic aorta according to claim 15, wherein the second stent meshing further includes an elliptical mesh or a mesh defining the contour of a hyperbolic paraboloid surface, at the base of the flange, so as to maintain the aperture made on the upper face of the arcuate central part of the main tubular endoprosthesis.

19. The fenestrated endoprosthesis of the thoracic aorta according to claim 15, wherein the first stent meshing mainly consists of a plurality of disjointed meshes, each extending over the entire circumference of the main tubular endoprosthesis in substantially sinusoidal or zigzag circumvolutions, the radial force of said meshing making it possible to press the main tubular endoprosthesis against the aortic wall and thus reduce the risk of endoleaks, and reinforce the anchoring of the endoprosthesis.

20. The fenestrated endoprosthesis of the thoracic aorta according to claim 15, wherein the first stent meshing includes at least one mesh attached to the second stent meshing, so as to prevent plications of the main tubular endoprosthesis.

21. The fenestrated endoprosthesis of the thoracic aorta according to claim 18, wherein the first stent meshing includes at least one mesh attached to the mesh of the base of the flange, so as to prevent plications of the main tubular endoprosthesis.

22. The fenestrated endoprosthesis of the thoracic aorta according to claim 15, wherein at least one of the ends of the main tubular endoprosthesis or the free end of the flange is an apparent meshing zone devoid of fabric.

23. The fenestrated endoprosthesis of the thoracic aorta according to claim 15, wherein the stent meshings are made of a shape memory alloy, preferably a Nickel-Titanium alloy, and the fabric covering the stent meshings is selected from the list defined by braided polyesters, braided Dacron, PTFE.

24. The fenestrated endoprosthesis of the thoracic aorta according to claim 15, wherein the fenestrated endoprosthesis comprises radiopaque markers, preferably disposed at the ends of the main tubular endoprosthesis and at the free end of the flange.

25. The fenestrated endoprosthesis of the thoracic aorta according to claim 15, wherein the first meshing projects onto the flange.

26. The fenestrated endoprosthesis of the thoracic aorta according to claim 15, wherein the second meshing projects onto the main tubular endoprosthesis.

27. The fenestrated endoprosthesis of the thoracic aorta according to claim 15, wherein a reinforcement extends along the convex face of the arcuate central part of the main tubular endoprosthesis, and preferably over the entire length of said main tubular endoprosthesis.

28. The fenestrated endoprosthesis of the thoracic aorta according to claim 27, wherein the reinforcement is made of nitinol or consists of suture wire on the fabric.
